(19) United States
(12) Patent Application Publication
(10) Pub. No.: US 2015/0374717 A1
(43) Pub. Date: Dec. 31, 2015

(54) CABAZITAXEL AND ITS USE FOR TREATING METASTATIC PROSTATE CANCERS

(71) Applicant: AVENTIS PHARMA S.A., Antony (FR)

(72) Inventor: Steven NEIBART, Bridgewater, NJ (US)

(21) Appl. No.: 14/844,480

(22) Filed: Sep. 3, 2015

Related U.S. Application Data
(63) Continuation of application No. PCT/EP2014/054156, filed on Mar. 4, 2014.

(30) Foreign Application Priority Data
Mar. 4, 2013 (EP) 13305243.1

(51) Int. Cl. A61K 31/573; A61K 31/337

(52) U.S. Cl. CPC A61K 31/573; A61K 31/337

(57) ABSTRACT

The present disclosure relates to the compound having the following formula (I):

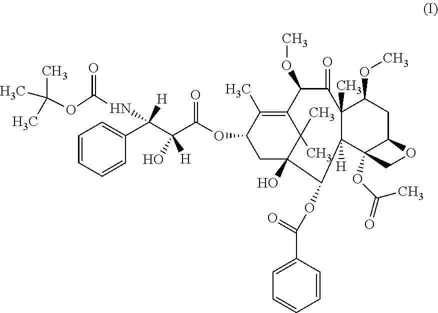 [SMILES: CO[C@H]1C(=O)[C@]2(C)[C@@H](OC)C[C@H]3OC[C@@]3(OC(C)=O)[C@H]2[C@H](OC(=O)c2ccccc2)[C@]2(O)C[C@H](OC(=O)[C@H](O)[C@@H](NC(=O)OC(C)(C)C)c3ccccc3)C(C)=C1C2(C)C]

which may be in base form or in the form of a hydrate or a solvate,
in combination with prednisone or prednisolone,
for its use for the treatment of castration resistant or hormone-refractory metastatic prostate cancer in patients not at risk of developing gastrointestinal disorders chosen from the group consisting of: gastrointestinal bleeding and perforation, gastritis, enterocolitis, neutropenic enterocolitis, colitis, intestinal obstruction, and ileus.

This application is a continuation of International Patent Application No. PCT/EP2014/054156, filed Mar. 4, 2014, which is incorporated herein by reference; and claims priority to European Application No. 13305243.1, filed Mar. 4, 2013.

The present invention concerns the use of cabazitaxel for treating metastatic prostate cancers. It also concerns a method of managing the risk of gastrointestinal disorders to allow an effective and safe use of cabazitaxel in the treatment of patients treated for castration resistant or hormone-refractory metastatic prostate cancer.

Prostate cancer affects a large proportion of the male population worldwide. It is the most frequently occurring cancer in men after lung cancer.

Prostate cancer is generally treated at the start by depriving the androgenic hormones, i.e. by surgical excision of the testicles (The Current State of Hormonal Therapy for Prostate Cancer CA Cancer J. Clin., May 2002; 52: 154-179), or by radiotherapy treatment (External beam radiation therapy for prostate cancer CA Cancer J. Clin., November 2000; 50: 349-375). Treatments with antiandrogens or hormone manipulations are associated with responses of short duration and without any improvement in the survival time.

Cabazitaxel is a semi-synthetic derivative of the natural taxoids 10-deacetylbaccatin III with potential antineoplastic activity. Cabazitaxel binds to and stabilizes tubulin, resulting in the inhibition of microtubule depolymerization and tumor cell proliferation. Unlike other taxane compounds, this agent is active in tumor models poorly or not sensitive to chemotherapy, including taxanes. In addition, cabazitaxel penetrates the blood-brain barrier.

Cabazitaxel has been developed and is registered for the treatment of patients with hormone refractory metastatic prostate cancer (HRPC) previously treated with a docetaxel containing regimen, in combination with prednisone or prednisolone. The recommended dose is 25 mg/m² administered as a 1-hour intravenous infusion every 3 weeks in combination with oral prednisone or prednisolone 10 mg administered daily throughout cabazitaxel treatment. It is available in vials of concentrate (sterile concentrate) and solvent for solution for infusion (60 mg).

It has been observed that the use of cabazitaxel for the treatment of metastatic prostate cancer may provoke some adverse reactions which may be fatal for the patients.

The aim of the present invention is to provide an efficient dosage regimen for the treatment of metastatic prostate cancer for patients having gastrointestinal disorders history.

The present invention relates to the compound having the following formula (I):

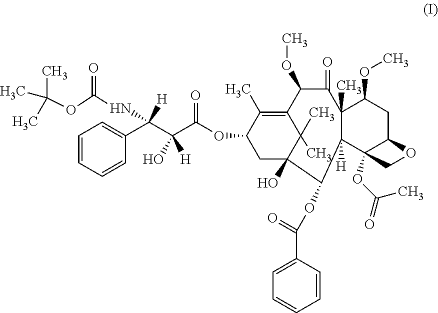 [SMILES: CO[C@H]1C(=O)[C@]2(C)[C@@H](OC)C[C@H]3OC[C@@]3(OC(C)=O)[C@H]2[C@H](OC(=O)c2ccccc2)[C@]2(O)C[C@H](OC(=O)[C@H](O)[C@@H](NC(=O)OC(C)(C)C)c3ccccc3)C(C)=C1C2(C)C]

which may be in base form or in the form of a hydrate or a solvate,

in combination with prednisone or prednisolone,

for its use for the treatment of castration resistant or hormone-refractory metastatic prostate cancer in patients not at risk of developing gastrointestinal disorders chosen from the group consisting of: gastrointestinal bleeding and perforation, gastritis, enterocolitis, neutropenic enterocolitis, colitis, intestinal obstruction, and ileus.

The present invention also relates to a method for treating castration resistant or hormone-refractory metastatic prostate cancer in patients not at risk of developing gastrointestinal disorders chosen from the group consisting of: gastrointestinal bleeding and perforation, gastritis, enterocolitis, neutropenic enterocolitis, colitis, intestinal obstruction, and ileus, said method comprising the administration of a pharmaceutically acceptable amount of the compound of formula (I) as defined above, which may be in base form or in the form of a hydrate or a solvate, in combination with prednisone or prednisolone.

In the present invention, the expression “castration resistant prostate cancer” is synonymous with the expression “hormone-refractory prostate cancer”.

The term “patient,” as used herein, includes both human and animals. In one embodiment, a patient is a human.

Gastrointestinal (GI) hemorrhage and perforation, ileus, colitis, including fatal outcome, have been reported in patients treated with cabazitaxel.

Symptoms such as abdominal pain and tenderness, fever, persistent constipation, diarrhoea, with or without neutropenia, may be early manifestations of serious gastrointestinal toxicity and should be evaluated and treated promptly. Cabazitaxel treatment delay or discontinuation may be necessary.

Caution is advised with treatment of patients most at risk of developing gastrointestinal complications: those with neutropenia, the elderly, concomitant use of NSAIDs, anti-platelet therapy or anti-coagulants, and patients with a prior history of pelvic radiotherapy, gastrointestinal disease, such as ulceration and GI bleeding.

According to a particular embodiment, the patients to be treated according to the invention do not display a prior history of neutropenia.

According to a particular embodiment, the patients to be treated according to the invention do not have a concomitant non-steroidal anti-inflammatory drugs (NSAIDs), anti-platelet or anti-coagulants therapy.

According to a particular embodiment, the patients to be treated according to the invention do not display a prior history of pelvic radiotherapy.

According to a particular embodiment, the patients to be treated according to the invention do not display a prior history of gastrointestinal diseases, such as ulceration and gastrointestinal bleeding.

According to a particular embodiment, the patients to be treated according to the invention are at least 50 years old, preferably 60-85 years old.

A particular group of patients for the present invention are patients who have been previously treated with docetaxel based regimen.

As mentioned above, within the context of the present invention, the gastrointestinal disorders may be gastrointestinal bleeding and perforations.

Upper gastrointestinal (UGI) bleeding is defined as bleeding originating proximal to the ligament of Treitz versus lower GI bleeding which is from the GI tract distal to the ligament of Treitz. Lower GI bleeding occurs less often than upper GI bleeding, with an annual incidence of approximately 20 per 100,000 and 47 per 100,000, respectively. Because of this, upper GI bleeds are the most common source for all causes of blood detected in the lower GI system, as blood must travel through the upper GI tract down to the lower GI system.

Grossly abnormal vital signs, including hypotension and tachycardia, or more subtle manifestations, such as a decreased pulse pressure or tachypnea, may signify significant bleeding.

As mentioned above, within the context of the present invention, the gastrointestinal disorders may be chosen from the group consisting of: gastritis, enterocolitis, neutropenic enterocolitis, and colitis, which all refer to different areas of inflammation within the GI tract.

Gastritis is inflammation of the stomach, enterocolitis, of the small intestine and colon, and colitis, of the colon.

Epigastric pain, nausea, and vomiting may be present with acute gastritis, but the most common presentation of gastritis is GI bleeding, ranging from occult blood loss in the stool to massive upper GI hemorrhage. Physical findings may be normal, may reflect only the GI bleeding, or may reflect a severe underlying associated illness.

The chief findings with colitis are abdominal cramps, diarrheal stools containing blood and mucopurulent material, fever, weight loss, and anemia. On sigmoidoscopy, the rectal mucosa is eroded and friable.

Neutropenic enterocolitis presents a spectrum of severity from mild self-limiting cecal inflammation to fulminant bowel wall necrosis with perforation. The clinical syndrome is typically characterized by a triad of diarrhea, abdominal pain, and fever in the setting of cytotoxic therapy-induced neutropenia. Abdominal distention, nausea, vomiting, and diffuse watery or bloody diarrhea are also commonly observed.

As mentioned above, within the context of the present invention, the gastrointestinal disorders may be intestinal obstruction or ileus.

Intestinal obstruction is the inability of the intestinal tract to allow for regular passage of food and bowel contents secondary to mechanical obstruction or adynamic ileus. Adynamic ileus (paralytic ileus) is more common but is usually self-limiting and does not require surgical intervention. Mechanical obstruction can be caused by either intrinsic or extrinsic factors and generally requires definitive intervention in a relatively short period of time to determine the cause and minimize subsequent morbidity and mortality.

Ileus and intestinal pseudo-obstruction designate clinical syndromes caused by impaired intestinal motility and are characterized by symptoms and signs of intestinal obstruction in the absence of a lesion-causing mechanical obstruction.

Ileus is a major cause of morbidity in hospitalized patients.

Cabazitaxel belongs to the taxoid family and has the formula (I) as mentioned above.

The chemical name of cabazitaxel is 4α-acetoxy-2α-benzoyloxy-5β,20-epoxy-1β-hydroxy-7β,10β-dimethoxy-9-oxo-11-taxen-13α-yl(2R,3S)-3-tert-butoxycarbonylamino-2-hydroxy-3-phenylpropionate. Cabazitaxel is synonymously known as (2α,5β,7β,10β,13α)-4-acetoxy-13-({(2R,3S)-3-[(tertbutoxycarbonyl)amino]-2-hydroxy-3-phenylpropanoyl}oxy)-1-hydroxy-7,10-dimethoxy-9-oxo-5,20-epoxytax-11-en-2-yl benzoate.

This compound and a preparative method thereof is described in WO 96/30355, EP 0 817 779 B1 and U.S. Pat. No. 5,847,170, which are hereby incorporated herein by reference. Cabazitaxel may be administered in base form (cf. above formula), or in the form of a hydrate. It may also be a solvate, i.e. a molecular complex characterized by the incorporation of the crystallization solvent into the crystal of the molecule of the active principle (see in this respect page 1276 of J. Pharm. Sci. 1975, 64(8), 1269-1288). In particular, it may be an acetone solvate, and, more particularly, may be the solvate described in WO 2005/02846. It may be an acetone solvate of cabazitaxel containing between 5% and 8% and preferably between 5% and 7% by weight of acetone (% means content of acetone/content of acetone+cabazitaxel×100). An average value of the acetone content is 7%, which approximately represents the acetone stoichiometry, which is 6.5% for a solvate containing one molecule of acetone. The procedure described below allows the preparation of an acetone solvate of cabazitaxel:

940 ml of purified water are added at 20±5° C. (room temperature) to a solution of 207 g of 4α-acetoxy-2α-benzoyloxy-5β,20-epoxy-1β-hydroxy-7β,10β-dimethoxy-9-oxo-11-taxen-13α-yl(2R,3S)-3-tert-butoxycarbonylamino-2-hydroxy-3-phenylpropionate at about 92% by weight in about 2 litres of acetone, followed by seeding with a suspension of 2 g of 4α-acetoxy-2α-benzoyloxy-5β,20-epoxy-1β-hydroxy-7β,10β-dimethoxy-9-oxo-11-taxen-13α-yl(2R,3S)-3-tert-butoxycarbonylamino-2-hydroxy-3-phenylpropionate isolated from acetone/water in a mixture of 20 ml of water and 20 ml of acetone. The resulting mixture is stirred for about 10 to 22 hours, and 1.5 litres of purified water are added over 4 to 5 hours. This mixture is stirred for 60 to 90 minutes, and the suspension is then filtered under reduced pressure. The cake is washed on the filter with a solution prepared from 450 ml of acetone and 550 ml of purified water, and then oven-dried at 55° C. under reduced pressure (0.7 kPa) for 4 hours. 197 g of 4α-acetoxy-2α-benzoyloxy-5β,20-epoxy-1β-hydroxy-7β,10β-dimethoxy-9-oxo-11-taxen-13α-yl(2R,3S)-3-tert-butoxycarbonylamino-2-hydroxy-3-phenylpropionate acetone containing 0.1% water and 7.2% acetone (theoretical amount: 6.5% for a stoichiometric solvate) are obtained.

According to a preferred embodiment, the compound of formula (I) is in the form of an acetone solvate.

Preferably, this acetone solvate contains between 5% and 8%, and preferably between 5% and 7%, by weight of acetone.

Cabazitaxel may be administered parenterally, such as via intravenous administration. A galenical form of cabazitaxel suitable for administration by intravenous infusion is that in which the cabazitaxel is dissolved in water in the presence of excipients chosen from surfactants, cosolvents, glucose or sodium chloride, etc. For example, a galenical form of cabazitaxel may be prepared by diluting a premix solution of cabazitaxel contained in a sterile vial (80 mg of cabazitaxel+2 ml of solvent+Polysorbate 80) with a sterile vial containing a solution of 6 ml of water and ethanol (13% by weight of 95% ethanol) in order to obtain 8 ml of a solution ready to be rediluted in a perfusion bag. The concentration of cabazitaxel in this ready-to-redilute solution is about 10 mg/ml. The perfusion is then prepared by injecting the appropriate amount of this ready-to-redilute solution into the perfusion bag containing water and glucose (about 5%) or sodium chloride (about 0.9%).

According to the present invention, cabazitaxel is administered in combination with prednisone or prednisolone, as two distinct pharmaceutical preparations.

This combination and use thereof for treating prostate cancer is in particular described in the international application WO2011/051894.

According to an advantageous embodiment, the compound of formula (I) is administered at a dose of between 15 and 25 mg/m², the prednisone or prednisolone being administered at a dose of 10 mg/day.

According to an embodiment, the compound of formula (I) is administered at a dose of 25 mg/m².

According to an embodiment, the compound of formula (I) is administered at a dose of 20 mg/m².

In some aspects of the invention, cabazitaxel may be administered by intravenous infusion at a dose of between 15 and 25 mg/m², this administration cycle of the antitumour agent being repeated at an interval of 3 weeks between each cabazitaxel administration, which interval may be prolonged by 1 to 2 weeks depending on the tolerance to the preceding cabazitaxel administration.

According to an advantageous embodiment, the prednisone or prednisolone is administered daily and the compound of formula (I) is administered every three weeks.

Preferably, the administration of the compound of formula (I) is repeated as a new cycle every 3 weeks.

According to an embodiment, for the administration of the compound of formula (I) the median number of cycles is 6.

The present invention also relates to the compound of formula (I) as defined above, which may be in base form or in the form of a hydrate or a solvate, in combination with prednisone or prednisolone, for its use for the treatment of castration resistant or hormone-refractory metastatic prostate cancer,

the compound of formula (I) being administered at a dose of between 15 and 25 mg/m², and the prednisone or prednisolone being administered at a dose of 10 mg/day, the administration of said compound being repeated as a new cycle every 3 weeks,

wherein, if the patient experiences gastrointestinal disorders during this administration cycle, then the administration of the compound is delayed and continued once the gastrointestinal disorders are resolved or completely stopped, said gastrointestinal disorders being chosen from the group consisting of: gastrointestinal bleeding and perforation, gastritis, enterocolitis, neutropenic enterocolitis, colitis, intestinal obstruction, and ileus.

The present invention also relates to a method for treating castration resistant or hormone-refractory metastatic prostate cancer comprising the administration of a pharmaceutically acceptable amount of the compound of formula (I) as defined above, which may be in base form or in the form of a hydrate or a solvate, in combination with prednisone or prednisolone,

the compound of formula (I) being administered at a dose of between 15 and 25 mg/m², and the prednisone or prednisolone being administered at a dose of 10 mg/day, the administration of said compound being repeated as a new cycle every 3 weeks,

wherein, if the patient experiences gastrointestinal disorders during this administration cycle, then the administration of the compound is delayed and continued once the gastrointestinal disorders are resolved or completely stopped, said gastrointestinal disorders being chosen from the group consisting of: gastrointestinal bleeding and perforation, gastritis, enterocolitis, neutropenic enterocolitis, colitis, intestinal obstruction, and ileus.

The present invention is based on the monitoring of the patient concerning the gastrointestinal disorders as mentioned above. Indeed, if the patient to be treated experiences any of these gastrointestinal disorders during the administration cycle, then the cancer treatment has to be discontinued. Once the gastrointestinal disorder is resolved, the cancer treatment may be continued. Depending on the patient's conditions, the cancer treatment may also stopped.

The combination cabazitaxel/prednisone or prednisolone is administered repeatedly according to a protocol that depends on the patient to be treated (age, weight, treatment history, etc.), which can be determined by a skilled physician. In one aspect of the invention, cabazitaxel is administered by perfusion to the patient according to an intermittent program with an interval between each administration of 3 weeks, which may be prolonged by 1 to 2 weeks depending on the tolerance to the preceding administration. The median number of cycles is 6. The prednisone or prednisolone may be administered daily, for example in the form of one dosage intake per day, throughout the duration of the treatment. The currently recommended dose is 25 mg/m² of cabazitaxel administered as a one-hour infusion and 10 mg per day of prednisone or prednisolone administered orally.

In some aspects of the invention, the patient to be treated has prostate cancer that is resistant to hormone therapy (i.e., hormone refractory) and has previously been treated with docetaxel. In some aspects, the patient has prostate cancer that progressed during or after treatment with docetaxel. In some aspects, the patient was previously treated with at least 225 mg/m² cumulative dose of docetaxel. In a particular aspect, the patient showed progression of their disease in the six months following hormone therapy or during docetaxel treatment or after docetaxel treatment. In another particular aspect, the patient showed progression of their disease in the three months following hormone therapy or after docetaxel treatment.

In some aspects of the invention, the patient to be treated has a measurable tumour and may show progression of the disease via a metastatic lesion of the viscera or of a soft tissue of at least 1 cm determined by MRI or by an axial tomographic scan (CT scan).

In some aspects of the invention, the patient to be treated has an unmeasurable tumour and may show an increase in the PSA level with three measurements at a 1-week interval or the appearance of new lesions.

In some aspects of the invention, the patient to be treated has undergone castration by orchidectomy or with LHRH agonists, elimination of the androgens or monotherapy with estramustine.

In a preferred aspect, the life expectancy of the patient to be treated should be at least 2 months.

In some aspects, the treatment does not include patients who have previously received mitoxantrone, or who have received less than 225 mg/m² of docetaxel, or who have undergone a radiotherapy that has eliminated more than 40% of the marrow, who have received a treatment within the 4 weeks preceding the test, who have a neuropathy or a stomatitis, involving the brain or the meninges, who have shown severe hypersensitivity to polysorbate or to prednisone, whose blood analysis shows an appreciable decrease in neutrophils, haemoglobin or platelets, an increase in bilirubin and/or liver enzymes and creatinine, or who have heart problems or an infection requiring antibiotics.

The present invention also relates to a method of providing the compound of formula (I) as defined above, which may be in base form or in the form of a hydrate or a solvate, in combination with prednisone or prednisolone, wherein said compound is provided along with information indicating that it is useful for treating patients with castration resistant or hormone-refractory metastatic prostate cancer, said patients being not at risk of developing gastrointestinal disorders chosen form the group consisting of: gastrointestinal bleeding and perforation, gastritis, enterocolitis, neutropenic enterocolitis, colitis, intestinal obstruction, and ileus.

Preferably, the compound of formula (I) is in the form of an acetone solvate.

According to a preferred embodiment, the information comprises printed matter, preferably a label, that advises that the compound of formula (I) is useful for treating patients suffering from castration resistant or hormone-refractory metastatic prostate cancer, said patients being not at risk of developing gastrointestinal disorders.

The present invention also relates to a method of managing the risk of gastrointestinal disorders to allow an effective and safe use of the compound of formula (I) as defined above, in the treatment of patients treated for castration resistant or hormone-refractory metastatic prostate cancer, said method comprising the following steps:
a) initially check the patients suffering from castration resistant or hormone-refractory metastatic prostate cancer;
b) if any gastrointestinal disorder is detected during the treatment with the compound of formula (I), then the treatment should be discontinued;
c) if, after a diagnosis of gastrointestinal disorders is excluded, treatment with the compound of formula (I) should be reinitiated, patients should be closely monitored.

According to a preferred embodiment, the compound of formula (I) is in the form of an acetone solvate.

Preferably, the present invention relates to the method as defined above of managing the risk of gastrointestinal disorders chosen from the group consisting of: gastrointestinal bleeding and perforation, gastritis, enterocolitis, neutropenic enterocolitis, colitis, intestinal obstruction, and ileus.

The present invention also relates to a method of managing the risk of gastrointestinal disorders to allow an effective and safe use of the compound of formula (I) as defined above, in the treatment of patients treated for castration resistant or hormone-refractory metastatic prostate cancer, said method comprising the following steps:
a) initially check the patients suffering from castration resistant or hormone-refractory metastatic prostate cancer;
b) if any abdominal pain and tenderness, fever, persistent constipation, and/or diarrhoea, with or without neutropenia, is detected during the treatment with the compound of formula (I), then the treatment should be discontinued;
c) if, after a diagnosis of gastrointestinal disorders is excluded, treatment with the compound of formula (I) should be reinitiated, patients should be closely monitored.

Preferably, the gastrointestinal disorders are chosen from the group consisting of: gastrointestinal bleeding and perforation, gastritis, enterocolitis, neutropenic enterocolitis, colitis, intestinal obstruction, and ileus.

The present invention also relates to a method of promoting the use of the compound of formula (I) as defined above, the method comprising the step of conveying to a recipient at least one message selected from the group consisting of:

(a) the compound of formula (I) should be prescribed to a patient who has not been diagnosed with gastrointestinal disorders;

(b) the compound of formula (I) should be prescribed to a patient who does not have a prior history of gastrointestinal disorders;

(c) the compound of formula (I) is contraindicated in patients with a prior history of gastrointestinal disorders; and

(d) if any abdominal pain and tenderness, fever, persistent constipation, and/or diarrhoea, with or without neutropenia, is detected during the treatment with the compound of formula (I), then the treatment should be discontinued.

Preferably, the gastrointestinal disorders are chosen from the group consisting of: gastrointestinal bleeding and perforation, gastritis, enterocolitis, neutropenic enterocolitis, colitis, intestinal obstruction, and ileus.

The present invention also relates to an article of manufacture comprising:
a) a packaging material;
b) prednisone or prednisolone;
c) the compound of formula (I) in the form of an acetone solvate; and
d) a label or package insert contained within the packaging material indicating that gastrointestinal disorders can occur, said gastrointestinal disorders being chosen from the group consisting of: gastrointestinal bleeding and perforation, gastritis, enterocolitis, neutropenic enterocolitis, colitis, intestinal obstruction, and ileus.

Preferably, the article of manufacture as defined above comprises a label or package insert contained within the packaging material indicating that:

i) the compound of formula (I) in the form of an acetone solvate is contraindicated in patients with a prior history of gastrointestinal disorders, said gastrointestinal disorders being chosen from the group consisting of: gastrointestinal bleeding and perforation, gastritis, enterocolitis, neutropenic enterocolitis, colitis, intestinal obstruction, and ileus, and
ii) if any abdominal pain and tenderness, fever, persistent constipation, and/or diarrhoea, with or without neutropenia, is detected during the treatment with the compound of formula (I) in the form of an acetone solvate, then the treatment should be discontinued.

The present invention also relates to a package comprising the compound of formula (I) as defined above and a label, said label comprising one or more messages that:

(a) the compound of formula (I) should not be prescribed to a patient who has been diagnosed with gastrointestinal disorders;

(b) the compound of formula (I) should not be prescribed to a patient who has a prior history of gastrointestinal disorders; and

(c) the compound of formula (I) is contraindicated in patients with a prior history of gastrointestinal disorders; and

(d) if any abdominal pain and tenderness, fever, persistent constipation, and/or diarrhoea, with or without neutropenia, is detected during the treatment with the compound of formula (I) in the form of an acetone solvate, then the treatment should be discontinued,

said gastrointestinal disorders being chosen from the group consisting of: gastrointestinal bleeding and perforation, gastritis, enterocolitis, neutropenic enterocolitis, colitis, intestinal obstruction, and ileus.

Preferably, in the package of the invention, the compound of formula (I) is in the form of an acetone solvate.

### EXAMPLES

### Example 1

This example relates to the treatment of a 59-year old patient with metastatic prostate cancer.

This patient initiated study therapy with intravenous cabazitaxel for 14 days: cabazitaxel (20 mg/m²) intravenously (day 1) every 3 weeks and prednisone (10 mg) PO daily, from day 1 continuously.

On the 15^{th} day of cabazitaxel (cycle 1), the patient complained of epigastric pain. Two days later, fibrogastroduodenoscopy showed acute ulcer in the small curvature with a touch of fibrin 8 mm and a biopsy was done. The patient was hospitalized for antisecretory and antiulcer therapy; bisphosphonates administration. Corrective treatment with esomeprazol 40 mg intravenous, drotaverin 40 mg 3 times per day, almagel A 1 tea spoons (dosing) four times a day, metoclopramide 10 mg 3 times per day, metronidazole 500 mg IV TID and ampicillin/sulbactam (Ampisid) IM 1.5 mg TID was administered.

Ten days after this antisecretory and antiulcer therapy, control fibrogastroscopy showed atrophic gastritis, epithelisation of the ulcerous defect, residual effects of complete erosions in antral area. Results of biopsy showed fragments of gastric mucosa with presence of the moderate linph-plasmocytic infiltrate.

The patient had fully recovered ten days after the onset of the epigastric pain. Then, the cabazitaxel/prednisone treatment was continued as planned.

### Example 2

This example relates to the treatment of a 68-year old patient with metastatic prostate cancer.

This patient initiated study therapy with intravenous cabazitaxel for 14 days: cabazitaxel (20 mg/m²) intravenously (day 1) every 3 weeks and prednisone (10 mg) PO daily, from day 1 continuously.

75 days after the first and 6 days after the most recent dose (cycle 4, day 6 of cabazitaxel), the patient was admitted to hospital for abdominal pain. A CT scan was performed, which showed right ureteric obstruction. The renal function was normal. Diagnosis of colitis (grade 3) was established.

Event was described as colonic spasm secondary to chemotherapy induced colitis. Treatment consisted of amoxicillin, oxycodone and paracetamol. No action was taken with study drug and the patient recovered from the colitis three days later.

79 days after the first and 10 days after the most recent dose (cycle 4, day 10 of cabazitaxel), the patient experienced abdominal pain/spasms leading to hospitalization. The results of previous CT/bone scans and ultrasound were inconclusive with regards to cause of spasms. Patient was treated with fentanyl citrate (Actiq) and hyoscine butylbromide (Buscopan). Patient underwent a colonoscopy, which did not show cause of the spasms; biopsy was taken (distal prostitis). The patient was then discharged and also received 5th, 6th and 7th cycle of the study medications with event of colonic spasm ongoing without change. During 7th cycle the event colonic spasm changed from grade 2 to grade 1 and was considered fully resolved.

### Example 3

This example relates to the treatment of a 78-year old male patient with metastatic prostate cancer, according the following dosage: cabazitaxel (25 mg/m²)+prednisone 10 mg daily+budesonide (9 mg daily).

The patient initiated study medication cabazitaxel intravenously (I.V.) every 3 weeks and budesonide orally once daily for the treatment of prostate cancer.

He also received prednisone 5 mg twice daily.

43 days after the first and 1 day after the most recent dose of study medication, the patient experienced slight diarrhea without action taken.

45 days after the first and 3 days after the most recent cabazitaxel treatment, the patient was taken by ambulance due to new episode of sternal pain, angina, dizziness and shortness of breath. Vital signs in ambulance showed blood pressure 75/60 and pulse 170. In the hospital, his vital signs showed blood pressure 100/75 and pulse 110. He was found to have supraventricular tachycardia (SVT) 8 ventricular extrasystoles (VES). On the same day, blood work showed white blood cell (WBC) 6.9 (normal 4-10), haemoglobin 7.3 (normal 8.5-11) and creatinine 94 (normal 60-110). The patient was treated with metoprolol and high dose verapamil. The diarrhea continued and increased with rectal bleeding noted. Six days later, a sigmoidoscopy showed severe colitis. Treatment with loperamide was given. The reported events were supraventricular tachycardia with angina (grade 3) and colitis (grade 2).

Then, the patient had no more episodes of SVT or diarrhea.

This patient experienced then several episodes of gastrointestinal disorders during which the cabazitaxel treatment was discontinued. Once these gastrointestinal disorders were resolved, the cabazitaxel treatment was continued.

### Example 4

This example relates to a 62-year-old male patient (with prostate carcinoma) who initiated treatment with study drug cabazitaxel (20 mg/m²) and prednisone (10 mg) (cycle 1). The most recent dose of cabazitaxel (39.4 mg) prior to onset of event was administered during cycle 7.

145 days after the first and 7 days after the most recent dose of cabazitaxel, the patient developed enterocolitis (grade 3). The patient presented to emergency department complaining of loose watery stools (diarrhoea), vomiting, rectal (PR) bleeding and abdominal pain. Lab test revealed blood pressure as 92/54 (units and reference unspecified) and body temp at 38.0° C. The patient reported no constipation but he was constipated with each previous chemotherapy. The patient had no relief of stomach cramps with defecation; it was constant lower abdominal cramp which was not relieved by vomiting or opening bowels. The patient denied fevers/chills. The patient was febrile on admission and his systolic blood pressure was 100 (units and normal range not provided). Sigmoidoscopy up to 8 cm due to pain, erythematous mucosa, blood proctitis. Patient was admitted with the plan to do colonoscopy. Patient was admitted to hospital and was treated aggressively. Two units of red blood cell transfusion was administered to the patient. Intravenous fluids and pantoprazole, metoclopramide hydrochloride, gentamicin sulfate, flucloxacillin, piperacillin sodium/tazobactam sodium (Tazocin), loperamide hydrochloride and clavulin were given as corrective treatments.

Patient had no neutropenia, urine was NAD and his IV antibiotics was ceased. Three days later, the patient recovered from the reported event. The patient was discharged home the same day. At the time of discharge his blood pressure had improved and his PR bleeding had stopped.

The cabazitaxel/prednisone treatment was then continued.

### Example 5

This example relates to the treatment of a 51-year old male patient with a metastatic carcinoma of the prostate, according the following dosage: cabazitaxel 20 mg/m² intravenously (day 1) every 3 weeks and prednisone 10 mg PO daily, from day 1 continuously.

During cycle 4 (cycle 4, day 5 of cabazitaxel), 75 days after the first and 5 days after the most recent dose of cabazitaxel, the patient was hospitalized for hypotension grade 2 with a blood pressure of 90/60 mm/Hg and gastritis grade 2. The patient also experienced nausea (grade 2) on the same day. A gastroduodenoscopy confirmed significant antrum gastritis. During hospitalization, the patient's WBC decreased to grade 3, which required isolation. WBC level was at 2720 (units was not provided) and at 1000 on the day after. A new SAE of leucocytopenia was reported.

Corrective treatments included 0.9% NaCl (sodium chloride) IV drip 1000 ml/day, pantoprazole (40 mg/day), ondansetron hydrochloride (8 mg daily) and Intravenous moxifloxacin HCl (400 mg/day), filgrastim. Gastroscopy was also performed for gastritis. The patient recovered from leukopenia three days after the corrective treatment and from the events of hypotension, nausea and gastritis one further day later.

The cabazitaxel/prednisone treatment was then continued.

### Example 6

This case involves a 64-year-old male patient with metastatic castration resistant prostate cancer previously treated with a docetaxel-containing regimen.

The treatment was as follows: cabazitaxel 20 mg/m² on day 1 every 3 weeks in combination with prednisone/prednisolone.

This patient initiated therapy with cycle 1 of cabazitaxel in combination with prednisone.

On day 4 (cycle 1), while he was still at the hospital, he developed abdominal distention. He was diagnosed with abdominal inflammation. A CT scan of abdomen-pelvis-chest was performed, revealing a paralytic ileus (grade 4). Corrective therapy administered included placement of nasogastric tube.

On day 7 of cycle 1, the patient was hypotensive with a blood pressure of 50/30 mmHg, pulse of 120 bpm and pulmonary ausculation revealed rhonchus, crackles and wheezing; oxygen saturation was 88%. An additional diagnosis of pneumonia (grade 4) was established. Corrective therapy administered included IV fluids, antibiotics, colloids and sedation.

One day later, this patient died due to paralytic ileus and pneumonia.

### Example 7

This example relates to a 70-year old male patient with metastatic castration resistant prostate cancer previously treated with a docetaxel-containing regimen.

The following treatment was followed: cabazitaxel 25 mg/m² on day 1 every 3 weeks in combination with prednisone/prednisolone.

The patient initiated therapy with cycle 1 of cabazitaxel 25 mg/m² in combination with prednisone 10 mg.

On day 9 of the first cycle, this patient presented with a complaint of diarrhea, more than 10 episodes with blood in feces. Laboratory data revealed an absolute neutrophil count of 0.01. Neutropenia started 8 days after most recent cycle of study treatment. He was hospitalized with a diagnosis of neutropenic colitis; colitis was grade 3 and neutropenia was grade 4. Corrective therapy administered included antibiotics and intravenous fluids. Corrective therapy of ceftazidime and IV solutions were given and the patient recovered from neutropenia 5 days later and from colitis 9 days later.

The treatment was interrupted (stopped temporarily) due to reported events.

### Example 8

This example involves a 77-year-old male patient patients with metastatic hormone refractory prostate cancer previously treated with a docetaxel-containing regimen.

The dosage regimen was as follows: cabazitaxel 25 mg/m² intravenous infusion over 1 hour on day 1 of each 3 week cycle as well as oral prednisone/prednisolone 10 mg daily.

The patient received chemotherapy with cycle 1 of cabazitaxel.

On the 5^{th} day of cycle 1, this patient presented to the emergency room with a complaint of diarrhea. Laboratory data revealed neutropenia. He was hospitalized with diagnosis of diarrhea (grade 2) and neutropenia (grade 3). Two days later, toxicity grade for neutropenia increased to grade 4. Six days later, neutropenia resolved and the patient recovered. A coproculture showed infection by clostridium difficile. An additional diagnosis of clostridium difficile infection (grade 3), prolonging the patient's hospitalization, was reported.

Corrective therapy administered included meropenem and metronidazole and then, the patient recovered six days later.

The treatment was stopped temporarily.

### Example 9

This example relates to an 81-year-old male patient with metastatic hormone refractory prostate cancer previously treated with a docetaxel-containing regimen.

The following treatment was applied: cabazitaxel 20 mg/m² intravenously every 3 weeks, in combination with oral prednisone or prednisolone 10 mg daily.

The patient received therapy with cabazitaxel 20 mg/m² and prednisolone 10 mg as mentioned above.

Four days after the second administration, this patient was hospitalized with a diagnosis of acute bowel obstruction. At the time of admission, no neutropenia was identified. The following day, laboratory data revealed a neutrophil count of 0.4.

Surgery was suggested, however, was declined by the patient's family. Corrective therapy administered included antibiotics and hydration, which was discontinued 4 days after the initial hospitalization and palliative care only was administered.

A final diagnosis of neutropenic colitis (grade 4) was established, leading to the patient's death 5 days after initial hospitalization. The last CT scan concluded that large bowel distension had improved with resolution of mural gas.

Cause of death was listed as neutropenic colitis, bowel obstruction and metastatic prostate carcinoma.

### Example 10

This example relates to a 76-year-old male patient with metastatic prostate cancer. The following treatment was followed: cabazitaxel 25 mg/m² intravenously (day 1) every 3 weeks and prednisone 10 mg PO daily, from day 1 continuously.

This patient received cycle 1 of cabazitaxel in combination with daily prednisone.

7 days after the first cycle of study drug, the patient arrived in the emergency room complaining of diarrhea grade 2. One day later, neutrophil count was noted to be 0.1×10⁹/L (normal 1.4-7.7) revealing neutropenia. Abdominal x-ray revealed colitis. Neutropenic colitis (grade 4) was diagnosed and the patient was admitted. Blood culture showed no growth after 5 days. The patient was discharged but he was readmitted two days later due to grade 1 fever. Stool analysis showed no Clostridium difficile antigen growth. Sputum analysis was negative for Influenza A and B viruses. The patient was treated with filgrastim and tobramycin and no change was made to study drug.

The event resolved three days after the readmission and the patient was discharged the same day.

The cabazitaxel/prednisone treatment was then continued.

### Example 11

This example relates to a 74-year-old male patient with metastatic castration resistant prostate cancer previously treated with a docetaxel-containing regimen.

This patient was treated with cabazitaxel 20 mg/m² on day 1 every 3 weeks in combination with prednisone/prednisolone.

Nine days after the last dose of study treatment, the patient was admitted to hospital because of colitis in effect of neutropenia caused by cabazitaxel. The patient presented fever and was dehydrated. Blood work up showed WBC at 2.41×10³ cells/mm³ (3.5×10³-10.5×10³), neutrophils at 1.12×10³ cells/mm³ (1.7×10³-8.0×10³), platelet count at 93×10³ cells/mm³ (NR:150×10³-450×10³) and CRP at 0.39 mg/dL (NR:<0.5). Neutropenic infection (colitis) was diagnosed. The patient received antibiotic: vancomycin 500 mg 6/6 hours, meronen 1 g 8/8 hours and he was hydrated.

Two days after his admission, the patient took ciprofloxacin 500 mg 12/12 hours and metronidazole 400 mg 8/8 hours.

The patient recovered two days after admission.

The cabazitaxel/prednisone treatment was then continued.

### Example 12

This example relates to a 73-year-old male patient with hormone refractory metastatic prostate cancer previously treated with a Taxotere containing regimen.

The following therapy dosage was used: cabazitaxel 25 mg/m² intravenously (day 1) every 3 weeks, plus prednisone 10 mg orally daily.

This patient received cycle 1 therapy with cabazitaxel 25 mg/m² intravenously once per 3 weeks and prednisone 10 mg by mouth daily for the treatment of hormone refractory metastatic prostate cancer.

This patient presented 13 days after receiving the first dose with a fever of 38.5° C. and grade 3 lower abdominal pain which resulted in the need for hospitalization.

He was previously treated for one episode of diarrhea and 2 bilious type episodes of vomiting. He was diagnosed with grade 4 enterocolitis, grade 4 febrile neutropenia, grade 4 renal failure, and grade 4 septic shock.

Corrective treatments of G-CSF, meropenem, vancomycin, amikacin, dopamine, and noradrenaline were given.

The patient expired 2 days after admission. Cause of death was septic shock secondary to neutropenic enterocolitis.

### Example 13

This example relates to a 71-year-old a male patient with metastatic hormone refractory prostate cancer previously treated with a docetaxel-containing regimen.

The therapy was as follows: the patient received cabazitaxel 25 mg/m² intravenous infusion over 1 hour on day 1 of each 3 week cycle as well as oral prednisone/prednisolone 10 mg daily.

The patient received chemotherapy with first cycle of cabazitaxel.

Mesenteriic, retroperitoneal, iliaco-caval and inguinal lymphadenopathy were seen. Ten days after, patient was hospitalized due to neutropenic enterocolitis and sepsis. Therapy with meropenem and metronidazole were given. Two weeks later, the patient had fever episodes and pasty stools again. Norovirus was detected in stool and blood cultures (peripheral and port) were positive.

The patient's condition declined and refused further treatment. The patient died with cause of death reported as progression of disease.

### Example 14

This example relates to a 75 year-old male patient with castration resistant prostate cancer previously treated with a docetaxel-containing regimen.

The following dosage regimen was used: cabazitaxel 25 mg/m² on day 1 every 3 weeks in combination with prednisone/prednisolone.

This patient received cycle 1 of cabazitaxel in combination with daily oral prednisone. Cycle 9 was administered.

232 days after the first and 14 days after most recent dose, patient experienced abdominal pain and neutropenic enteritis. The patient was admitted for G3 peritonitis. Corrective treatment included conventional appendectomy, ertapenem, and metronidazole. The peritonitis was likely due to an intestinal (appendiceal) perforation.

Nine days after his hospitalization, the patient recovered from this event and was discharged.

For this patient, the treatment was permanently discontinued.

### Example 15

This example relates to a 63-year-old male patient with metastatic castration resistant prostate cancer not pretreated with chemotherapy. The following therapy was applied: cabazitaxel 25 mg/m² intravenously (day 1) every 3 weeks. Prednisone 10 mg PO daily, from day 1 continuously.

108 days after the first administration of cabazitaxel, the patient was presented with abdominal pain and grade 1 diarrhea and took loperamide (Immodium) by himself.

During cycle 6, 111 days after the first administration of study medication and 6 days after the last administration of study medication the patient experienced Grade 1 rectal bleeding and was hospitalized for further examination. Video coloscopy revealed probable infectious colitis. On the same day, the patient was treated with metronidazole (flagyl) 500 mg; 3 times a day and ciprofloxacin (Ciflox) 500 mg; twice daily for 5 days. Patient also received paracetamol (Doliprane), aluminium hydroxide/magnesium trisilicate (Gaviscon), esomeprazole (Inexium) and smectite (Smecta) as a corrective treatment during hospitalization.

The patient recovered 4 days later and was discharged from the hospital. Finally, the bacteriological tests returned negative for infectious colitis.

For this patient, the treatment was permanently discontinued.

### Example 16

This example relates to a 67-year-old male patient with metastatic castration resistant prostate cancer not pretreated with chemotherapy, having the following therapy: cabazitaxel 25 mg/m² intravenously (day 1) every 3 weeks and prednisone 10 mg PO daily, from day 1 continuously.

The patient initiated therapy with cabazitaxel 25 mg/m² in combination with prednisolone 10 mg.

On day 17 of cycle 1, this patient presented to the emergency room with a complaint of blood tinged vomiting. Initially, a suspicion of ulcus ventriculi (grade 3) was established, leading to his hospitalization. The follow-up report confirms the final diagnosis of gastritis (grade 3). Corrective therapy administered included intravenous fluids and pantoprazole. The patient recovered with no further gastrointestinal symptoms three days later. Gastroscopy was not performed since patient has not had further signs of gastritis.

One day later, a urinary tract infection grade 3 was diagnosed via a positive urine culture. The patient was treated with clavulin, pivmecillinam, piperacillin/tazobactam, metronidazole, gentamicin, cefuroxime, morphine and transexamic acid and the event resolved 10 days later.

Five days after the hospitalization, the patient developed DIC (grade 4) with decreased platelets and increased INR of 1.6. Corrective measures included fresh frozen plasma (FFP) and IV antibiotics. The patient was also transfused with whole blood and platelets on the same day. Life threatening and hospitalization are serious criteria for this event. CT scan of the head revealed subarachnoid bleeding of the right and left posterior horns of the ventricle. An event of SAH (subarachnoid hemorrhage) grade 2 was reported. Corrective measures consisted of fresh frozen plasma. The seriousness criteria for SAH was that it required a prolonged hospitalization. The patient recovered with sequelae (somnolence) from the event of SAH 6 days after the hospitalization.

The cabazitaxel/prednisone treatment was then continued.

### Example 17

This example relates to a 73-year-old male patient with metastatic castration resistant prostate cancer not pre-treated with chemotherapy. The following therapy was applied: cabazitaxel 20 mg/m² intravenously (day 1) every 3 weeks, prednisone 10 mg PO daily, from day 1 continuously.

42 days after the first and 21 days after the most recent dose of cabazitaxel (cycle 2 day 21), the patient was admitted to the hospital due to severe abdominal pain, nausea and vomiting. Thorax x-ray suggested perforation of ulcer. The patient underwent emergency surgery, which confirmed the diagnosis. The event was considered as life-threatening. Perforation was sutured and the patient was treated with intravenous (IV) antibiotics (metronidazole, ciprofloxacin and tazobactam). The patient recovered seven days after his hospitalization and he was discharged from hospital.

For this patient, the treatment was permanently discontinued.

### Example 18

This example relates to a 74-year-old male patient with metastatic hormone refractory prostate cancer previously treated with a docetaxel-containing regimen.

The following dosage regimen was applied: cabazitaxel 25 mg/m² intravenously every 3 weeks, in combination with oral prednisone or prednisolone 10 mg daily.

On day 16 of cycle 1 of cabazitaxel, this patient started to experience epigastric pain. Four days later, he was hospitalized due to melena and hematochesia for the event of duodenal ulcers grade 1. Acute gastroscopy was performed, which showed multiple duodenal ulcers. Intravenous (IV) proton-pump inhibitor (PPI) was started. The patient was transfused with 3 units of red blood cells. He also received parenteral then oral PPI (proton-pump inhibitor). The melena stopped and patient recovered.

On day 26 of cabazitaxel of cycle 1, the patient developed acute cystitis (grade 1). He was hospitalized once again the day after due to fever caused by acute cystitis. He was started on ciprofloxacin and the fever subsided. The patient also had anemia (not reported as SAE), which was due to previous gastritis and was treated with one unit of RBC concentrate. The event of acute cystitis recovered 8 days after the second hospitalization and the patient was discharged on the same day.

For this patient, the cancer treatment was then continued.

### Example 19

This example relates to a 77-year-old male patient with metastatic castration resistant prostate cancer previously treated with a docetaxel-containing regimen.

The therapy involved the administration of cabazitaxel on day 1 every 3 weeks in combination with prednisone.

About 128 days after the first dose and 20 days after receiving the last dose of cabazitaxel, the patient repeatedly vomited “coffee ground” like and stool looked black (melena). Lab test revealed that the patient was anaemic. The patients haemoglobin was 5.12 g/dl (reference range: 13.0-16.0), haematocrit was 16.33% (reference: 42-52), thrombocyte count was 193×10⁶/mcl (reference range: 150-450). The patient was diagnosed with life threatening event of digestive haemorrhage (grade 4) and was admitted to hospital.

During hospitalization, the patient received treatment with pantoprazole and erythrocyte concentrates. Prednisone treatment was temporarily stopped.

Patient recovered from the event and was discharged 9 days after his hospitalization.

For this patient, the treatment was temporarily stopped.

### Example 20

This example relates to a 66-year-old male patient with metastatic castration resistant prostate cancer previously treated with docetaxel-containing regimen.

The following therapy was applied: cabazitaxel 25 mg/m² on day 1 every 3 weeks in combination with prednisolone.

17 days after the first and the most recent dose of study medication, blood was detected in patient's stool and the patient was hospitalised. Patient was diagnosed with life threatening event of intestinal haemorrhage, grade 4. Patient's condition became worst after chemotherapy for prostate carcinoma.

On admission, haemoglobin was 59 g/l (normal units not specified) and haematocrit was 19% (normal units not specified). Patient did not receive any anticoagulation therapy. Therapy with cabazitaxel was withdrawn. The patient was treated with haemostatic therapy, anti-anaemic therapy with packed red blood cells, blood plasma and fluid maintenance. After hemodynamic stabilization was done, esophagogastroduodenoscopy was done with finding of incompetent cardiac sphincter, chronic mixed gastritis and anaemia of mucous membranes of proximal regions of gastrointestinal tract. Ultrasound investigation of abdomen cavity showed echo graphic picture of hepatomegaly, diffuse changes of liver and pancreas. Free fluid was not found. After intensive care patient condition stabilized with haemoglobin as 72 g/l (normal units not specified), haematocrit was 19.5% (normal units not specified) and platelets as 142×10⁹ (normal units not specified).

Three days after his hospitalization, conservative treatment continued. After the treatment, patient's condition improved without recurrence of bleeding. Two days later X-ray thorax showed emphysema and pneumosclerosis and enlargement of heart shadow to the left. Thirteen days after his hospitalization, platelet were 122 (units not specified, 180-320 thousand in 1 mcl). Patient recovered from the event two days later and was discharged from hospital the same day.

For this patient, the treatment was permanently discontinued.

### Example 21

This example relates to a 70-year male patient with metastatic castration resistant prostate cancer not pretreated with chemotherapy.

The following dosage regimen was applied: cabazitaxel 20 mg/m² intravenously (day 1) every 3 weeks and prednisone 10 mg PO daily, from day 1 continuously.

23 days after the first dose of study medication and 2 days after the most recent dose (cycle 2, day 2 of cabazitaxel), the patient experienced abdominal pain and was constipated.

One day later, the patient went to the hospital and had enema. X-ray showed that the patient was impacted and then sent to home.

On the next day, the patient came back to hospital. CT scan showed a 15 cm dilation in his small bowel. The pain persisted; therefore the patient came into the hospital ER to be admitted. CT showed distended loops of small bowel with a transient point in the left lower quadrant, which supported the diagnosis of small bowel obstruction grade 4, most likely secondary to adhesions. The patient started on conservative management, which consisted of bowel rest, nasogastric suctioning, IV fluids and pain management. During his hospital stay, the patient developed febrile neutropenia for what he started on IV antibiotics and filgrastim (Neupogen).

The patient was discharged 10 days after the second hospitalization and he had recovered one day after his discharge.

For this patient, the treatment was temporarily stopped.

### Example 22

This example relates to a 71 year-old male patient with metastatic castration resistant prostate cancer previously treated with a docetaxel-containing regimen.

The following dosage regimen was applied: cabazitaxel 20 mg/m² on day 1 every 3 weeks in combination with prednisolone.

During cycle 9, 181 days after the first and 13 days after the most recent dose of cabazitaxel, the patient experienced abdominal pain and was hospitalized for the event of small intestinal obstruction (grade 3). CT scan was showed high grade small bowel obstruction at the mid ileal level in the right flank. A sharp transition point, including a closed loop of small bowel and apparent twisting in keeping with a high grade mechanical small bowel obstruction, which could be probably due to adhesion. Within the proximal to mid jejunum, a 10 cm length segment of apparent thick walled jejunum. The patient was given with morphine for abdominal pain.

Three days later, laproscopy was performed and event was recovered the same day. Patient was discharged 6 days later.

For this patient, the treatment was temporarily stopped.

### Example 23

This example relates to a 79-year male patient with metastatic castration resistant prostate cancer previously treated with a docetaxel-containing regimen.

The following dosage regimen was applied: cabazitaxel 25 mg/m² on day 1 every 3 weeks in combination with prednisone.

85 days after the first dose of study medication and 1 day after the most recent dose of study treatment, the patient experienced hematuria grade 3 and red blood loss per anum grade 1.

Four days later, the patient was hospitalized due to hematuria and red blood loss per anum since 4 days. The patient was treated tranexamic acid (Exacyl) as corrective treatment for hematuria. No corrective treatment was taken for red blood loss per anum. Hematuria and blood loss per anum were caused by the combination of more fragile rectum as a result of prior radiotherapy for prostate cancer and thrombopenia which was caused by chemotherapy.

Ultrasound kidneys and bladder showed cyst in left kidney. One day after his hospitalization, left colonoscopy was abnormal and showed diverticulosis of sigmoid. Bleeding stopped spontaneously during hospitalization. The patient recovered from red blood loss per anum and hematuria two days after the hospitalization and discharged on the same day.

95 days after the first dose of study medication and 11 days after the most recent dose of study treatment, the patient experienced general weakness. Five days later, the patient experienced grade 3 pathologic fracture lumbar spine 1. On the next day, the patient came to emergency room due to general weakness (grade 3), worsening lower back pain, nausea, vomiting, anorexia and dyspnoea and was hospitalized. As corrective treatment the patient received tramadol hydrochloride (Contramal), diclofenac sodium (Diclofenac), paracetamol (Perfusalgan), therapeutic radiopharmaceuticals (Radiation therapy) and Sodium chloride (NaCl 0.9 percent). The following day, the patient underwent CT scan of lumbar spine and showed bone metastases and pathologic fracture lumbar spine 1. Fracture was not due to progressive disease. Pain medication was adapted.

Arterial blood gas sample was taken and abnormal values which indicate the general weakness of patient were summarized as follows: pH: 7.49 pH (normal 7.38-7.42), pco2: 24 mm hg (normal 38-42), base excess: −3.5 mmol/l (normal −2 to +3), hematocrite: 37% (normal 40-54), glucose: 103 mg/dl (normal 65-95), sodium: 135 mmol/l (normal 136-145) and lactate: 2.4 mmol/l (normal less than 1.3).

During hospitalization, extreme progression was seen with increasing LDH in lab and general deterioration of condition of the patient. Palliative sedation was started on patient's demand. The patient died due to disease progression 117 days after the first dose of study medication.

### Example 24

This example relates to a 68-year-old male patient with metastatic hormone refractory prostate cancer previously treated with a docetaxel-containing regimen.

The applied dosage regimen was as follows: cabazitaxel 25 mg/m² intravenous infusion over 1 hour on day 1 of each 3 week cycle as well as oral prednisone 10 mg daily.

Ten days after most recent administration (143 days after the administration of the first dose), patient developed G3 colitis and was hospitalized due to diarrhea. An ileocoloscopy showed colitis. Cause was unknown, but probably due to radiation therapy.

About a month after hospitalization, patient died due to unknown cause. Colitis was ongoing at the time of patient's death.

### Example 25

This example relates to a 71-year male patient with metastatic castration resistant prostate cancer not pre-treated with chemotherapy.

The following dosage regimen was applied: cabazitaxel 20 mg/m² intravenously (day 1) every 3 weeks and prednisone 10 mg PO daily, from day 1 continuously.

214 days after the administration of the first dose, the patient complained about vomiting and abdominal pain. The patient visited the physician who diagnosed the patient with ileus.

215 days after the first dose of study medication and 6 days after the most recent dose of cabazitaxel, the patient visited hospital and further diagnostics were performed with the initial diagnosis of paralytic DD mechanical ileus. A CT scan of thorax/abdomen/pelvis was performed and showed a mechanical obstruction of ileum due to a new lesion of unclear cause (adhesion DD tumor). Event reported as mechanical ileus due to unclear cause grade 2. Admission and surgery was recommended, but patient declined admission and decided to wait until stronger symptoms. The event of ileus resolved 13 days later (the event was likely due to progression of the disease).

What is claimed is:

1. A method for treating castration resistant or hormone-refractory metastatic prostate cancer, comprising administering to a patient in need thereof a therapeutically effective amount of a compound of formula (I): 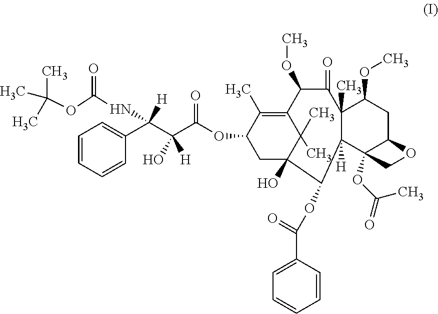 [SMILES: CO[C@H]1C(=O)[C@]2(C)[C@@H](OC)C[C@H]3OC[C@@]3(OC(C)=O)[C@H]2[C@H](OC(=O)c2ccccc2)[C@]2(O)C[C@H](OC(=O)[C@H](O)[C@@H](NC(=O)OC(C)(C)C)c3ccccc3)C(C)=C1C2(C)C]
which may be in base form or in the form of a hydrate or a solvate,
in combination with prednisone or prednisolone,
wherein the patient is not at risk of developing a gastrointestinal disorder chosen from the group consisting of: gastrointestinal bleeding and perforation, gastritis, enterocolitis, neutropenic enterocolitis, colitis, intestinal obstruction, and ileus.

2. The method according to claim 1, wherein the patient does not display a prior history of neutropenia.

3. The method according to claim 1, wherein the patient does not have a concomitant non-steroidal anti-inflammatory drugs (NSAIDs), anti-platelet or anti-coagulants therapy.

4. The method according to claim 1, wherein the patient does not display a prior history of pelvic radiotherapy.

5. The method according to claim 1, wherein the patient does not display a prior history of a gastrointestinal disease.

6. The method according to claim 1, wherein the patient is at least 50 years old.

7. The method according to claim 1, wherein the patient has been previously treated with a docetaxel based regimen.

8. The method according to claim 1, wherein the compound of formula (I) is in the form of an acetone solvate.

9. The method according to claim 8, wherein the acetone solvate contains between 5% and 8% by weight of acetone.

10. The method according to claim 1, wherein the compound of formula (I) is administered at a dose of between 15 and 25 mg/m², and the prednisone or prednisolone is administered at a dose of 10 mg/day.

11. The method according to claim 10, wherein the compound of formula (I) is administered at a dose of 25 mg/m².

12. The method according to claim 10, wherein the compound of formula (I) is administered at a dose of 20 mg/m².

13. The method according to claim 1, wherein the administration of the compound of formula (I) is repeated as a new cycle every 3 weeks.

14. The method according to claim 13, wherein the median number of cycles is 6.

15. A method for treating castration resistant or hormone-refractory metastatic prostate cancer, comprising administering to a patient in need thereof a therapeutically effective amount of a compound of formula (I): 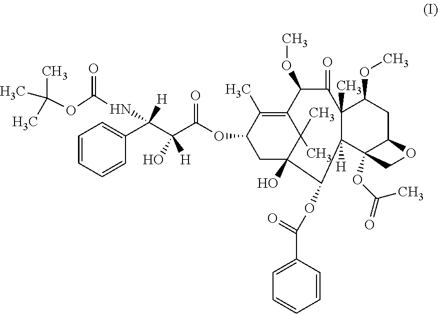 [SMILES: CO[C@H]1C(=O)[C@]2(C)[C@@H](OC)C[C@H]3OC[C@@]3(OC(C)=O)[C@H]2[C@H](OC(=O)c2ccccc2)[C@]2(O)C[C@H](OC(=O)[C@H](O)[C@@H](NC(=O)OC(C)(C)C)c3ccccc3)C(C)=C1C2(C)C]
which may be in base form or in the form of a hydrate or a solvate,
in combination with prednisone or prednisolone,
wherein the compound of formula (I) is administered at a dose of between 15 and 25 mg/m², and the prednisone or prednisolone is administered at a dose of 10 mg/day, and the administration of said compound is repeated as a new cycle every 3 weeks,
wherein, if the patient experiences gastrointestinal disorders during this administration cycle, then the administration of the compound is delayed and continued once the gastrointestinal disorders are resolved or completely stopped, said gastrointestinal disorders being chosen from the group consisting of: gastrointestinal bleeding and perforation, gastritis, enterocolitis, neutropenic enterocolitis, colitis, intestinal obstruction, and ileus.

16. An article of manufacture comprising:
a) a packaging material;
b) prednisone or prednisolone;
c) the compound of formula (I) in the form of an acetone solvate; and
d) a label or package insert contained within the packaging material indicating that gastrointestinal disorders can occur, said gastrointestinal bleeding and perforation, gastritis, enterocolitis, neutropenic enterocolitis, colitis, intestinal obstruction, and ileus.

17. The article of manufacture of claim 16, comprising a label or package insert contained within the packaging material indicating that:
i) the compound of formula (I) in the form of an acetone solvate is contraindicated in patients with a prior history of gastrointestinal disorders, said gastrointestinal disorders being chosen from the group consisting of: gastrointestinal bleeding and perforation, gastritis, enterocolitis, neutropenic enterocolitis, colitis, intestinal obstruction, and ileus, and
ii) if any abdominal pain and tenderness, fever, persistent constipation, and/or diarrhoea, with or without neutropenia, is detected during the treatment with the compound of formula (I) in the form of an acetone solvate, then the treatment should be discontinued.

18. A package comprising the compound of formula (I) of claim 1 and a label, said label comprising one or more messages that:
(a) the compound of formula (I) should not be prescribed to a patient who has been diagnosed with gastrointestinal disorders;
(b) the compound of formula (I) should not be prescribed to a patient who has a prior history of gastrointestinal disorders; and
(c) the compound of formula (I) is contraindicated in patients with a prior history of gastrointestinal disorders; and
(d) if any abdominal pain and tenderness, fever, persistent constipation, and/or diarrhoea, with or without neutropenia, is detected during the treatment with the compound of formula (I) in the form of an acetone solvate, then the treatment should be discontinued,
said gastrointestinal disorders being chosen from the group consisting of: gastrointestinal bleeding and perforation, gastritis, enterocolitis, neutropenic enterocolitis, colitis, intestinal obstruction, and ileus.

19. The package according to claim 18, wherein the compound of formula (I) is in the form of an acetone solvate.
